(19) United States
(12) Patent Application Publication
(10) Pub. No.: US 2012/0136456 A1
(43) Pub. Date: May 31, 2012

(54) CERAMIC FOAM ARTIFICIAL BONE

(75) Inventors: Hadar BETTER, Tel Aviv (IL); Ricardo OSIROFF, Ganei-Tikva (IL); Yossi GROSS, Moshav Mazor (IL)

(73) Assignee: RAINBOW MEDICAL LTD., Herzliya (IL)

(21) Appl. No.: 13/305,301

(22) Filed: Nov. 28, 2011

Related U.S. Application Data
(60) Provisional application No. 61/417,515, filed on Nov. 29, 2010.

(51) Int. Cl. A61F 2/28; B65B 55/00; B29C 67/00; C04B 35/64; B05D 5/00

(52) U.S. Cl. 623/23.51; 623/23.57; 264/332; 427/243; 264/239; 53/428

(57) ABSTRACT

A sterile artificial bone product is provided. The sterile artificial bone product comprises a sintered porous ceramic foam material having a density of 0.3 to 1.5 g/cc, and a porosity of 40 to 95%, pores of the material having a pore diameter distribution of 10-500 microns. Additionally, a bioactive compound is coupled to the ceramic foam material. Other embodiments are also described.

### CROSS-REFERENCES TO RELATED APPLICATIONS

The present application claims the priority of U.S. Provisional Application 61/417,515 to Better et al., entitled, “Ceramic foam artificial bone,” filed Nov. 29, 2010, which is incorporated herein by reference.

### FIELD OF THE APPLICATION

Applications of the present invention relate generally to prosthetics, and particularly to artificial bones and bone grafts.

### BACKGROUND OF THE APPLICATION

Artificial bones and bone grafting materials are often used to fill voids in human bones, provide mechanical support, and enhance biological repair of bone fractures and defects. Damaged native bone can be replaced with a bone substitute material made, for example, with various ceramics or metallic alloys.

Ceramic foam is foam made from a wide range of ceramic materials. Ceramic foams can be used for a wide range of applications including biomedical applications.

### SUMMARY OF APPLICATIONS

In some applications, a bone product such as an artificial bone and/or a material for use in a bone grafting procedure is provided. The artificial bone and bone grafting material typically comprises highly porous sintered ceramic foam. For some applications, the sintered porous ceramic foam is shaped to define at least a portion of a bone structure and is configured to be used as an artificial bone. Typically, the artificial bone is shaped to define a plurality of channels, allowing for blood vessels to grow within the channels. Typically, the ceramic foam comprises a growth factor such as bone morphogenetic protein, which induces bone and cartilage formation.

For some applications, ceramic foam shapes and/or ceramic foam pellets are manufactured as described in U.S. Pat. No. 6,602,449, U.S. Pat. No. 7,306,762 and U.S. Pat. No. 6,869,563. For some applications, these ceramic foams are adapted for use as artificial bones and/or bone grafting materials. For example, bioactive substances such as growth factors, e.g., bone morphogenetic proteins, are added to these ceramic foams. Additionally, these ceramic foams comprise an open-pore microstructure allowing for blood vessels to grow within the artificial bones and bone grafts. Further additionally, following manufacturing, the ceramic foams are sterilized and packaged in a sterile packaging. For applications in which these ceramic foams are configured for use as an artificial bone, the ceramic foams are machined into a bone structure following manufacturing thereof.

There is therefore provided, in accordance with some applications of the present invention, a sterile artificial bone product including:

a sintered porous ceramic foam material having a density of 0.3 to 1.5 g/cc, and a porosity of 40 to 95%, pores of the material having a pore diameter distribution of 10-500 microns; and a bioactive compound coupled to the material.

For some applications, the ceramic foam material is bone shaped.

For some applications, the ceramic foam material is a generally-rigid solid, having a volume of 0.25-1000 cc.

For some applications, the sintered porous ceramic foam material includes alumina.

For some applications, the sintered porous ceramic foam material includes alumina silicate.

For some applications, the sintered porous ceramic foam material includes a ceramic selected from the group consisting of zirconia, alumina zirconate, and alumina titanate.

For some applications, the bone product includes a compound selected from the group of fillers consisting of ceramic fibers, polymeric fibers, and particulate fillers.

For some applications, the bone product is shaped to provide a plurality of channels, at least some of the channels having a diameter selected from the group consisting of: 5-100 microns, 100-500 microns and 0.5-10 mm.

For some applications, the bioactive compound includes a substance selected from the group consisting of: a growth factor, an antibiotic, an antibacterial molecule, an antiviral agent, and a hormone.

For some applications, the bone product includes an adhesive promoter

For some applications, the bone product includes a surface modifier.

There is further provided, in accordance with some applications of the present invention, a sterile artificial bone product, including:

a material including sintered porous ceramic foam pellets having a density of 0.05 to 0.5 g/cc, and a porosity of 70 to 98%, pores of the material having a pore diameter distribution of 10-1000 microns; and

a bioactive compound coupled to the material.

For some applications, the sintered porous ceramic foam pellet is bone shaped.

For some applications, the material is a generally-rigid solid, having a volume of 0.25-1000 cc.

For some applications, the sintered porous ceramic foam includes alumina.

For some applications, the sintered porous ceramic foam pellet includes alumina silicate.

For some applications, the sintered porous ceramic foam pellet includes a ceramic selected from the group consisting of zirconia, alumina zirconate, and alumina titanate.

For some applications, the bone product includes a compound selected from the group of fillers consisting of ceramic fibers, polymeric fibers, and particulate fillers.

For some applications, the bone product is shaped to provide a plurality of channels, at least some of the channels having a diameter selected from the group consisting of: 5-100 microns, 100-500 microns and 0.5-10 mm.

For some applications, the bioactive compound includes a substance selected from the group consisting of: a growth factor, an antibiotic, an antibacterial molecule, an antiviral agent, and a hormone.

For some applications, the bone product includes an adhesive promoter.

For some applications, the bone product includes a surface modifier.

For some applications, the bone product includes a bone grafting material selected from the group consisting of: beta tricalcium phosphate and hydroxyapatite.

There is still further provided, in accordance with some applications of the present invention, a method for producing an artificial bone product, the method including:

providing a porous ceramic foam material having a density of 0.3 to 1.5 g/cc, and a porosity of 40 to 95%, pores of the material having a pore diameter distribution of 10-500 microns; and
sintering the ceramic foam material into a bone-shaped mold.

For some applications, sintering the ceramic foam material includes sintering the ceramic foam material into a mold of volume 0.25-1000 cc.

For some applications, providing the ceramic foam material further includes providing a ceramic foam material including a compound selected from the group of fillers consisting of ceramic fibers, polymeric fibers, and particulate fillers.

For some applications, the method includes creating channels in the ceramic foam material by heating the ceramic foam material sufficiently to destroy the fibers in the ceramic foam material.

There is additionally provided, in accordance with some applications of the present invention, a method for producing an artificial bone product, the method including:

identifying a desired bone shape; and

machining into the desired bone shape a sintered porous ceramic foam material made from ceramic foamed pellets, the foam material having a density of 0.3 to 1.5 g/cc, and a porosity of to 95%, pores of the material having a pore diameter distribution of 10-500 microns.

For some applications, the method includes coating the desired bone shape after the machining.

There is yet additionally provided, in accordance with some applications of the present invention, a method for producing an artificial bone product, the method including:

providing porous ceramic foam pellets having a density of 0.05 to 0.5 g/cc, and a porosity of 70 to 98%, pores of the material having a pore diameter distribution of 10-1000 microns;

sintering the ceramic foam pellets into an artificial bone product; and

packaging the artificial bone product in sterile packaging.

### DETAILED DESCRIPTION OF APPLICATIONS

A bone product comprising a ceramic foam material is provided in accordance with some applications of the present invention. The ceramic foam material may be provided in a raw form, for subsequent shaping into a desired shape, it may be pre-shaped in a desired shape (“ceramic foam shapes”), and/or it may be provided in the form of ceramic foam pellets.

In some applications, a sterile artificial bone structure comprising a sintered highly porous ceramic foam material is provided. Typically, the sintered porous ceramic foam shapes comprise a ceramic material such as, alumina, zirconia, alumina zirconate, alumina titanate, and/or alumina silicate. The ceramic foam material typically has a density of 0.3 to 1.5 g/cc, and a porosity of 40 to 95%, the pores having a pore diameter distribution of 10-500 microns.

For applications in which ceramic foam pellets are used, the ceramic foam pellets typically have a density of 0.05 to 0.5 g/cc and a porosity of 70 to 98%, the pores having a pore diameter distribution of 10-1000 microns.

Typically, techniques and ceramic foam materials described in one or more of the following patents are practiced and used in combination with materials and methods described herein: U.S. Pat. No. 6,602,449, U.S. Pat. No. 7,306,762 and U.S. Pat. No. 6,869,563, which are incorporated herein by reference.

For some applications, at least one ceramic foam as described in U.S. Pat. No. 6,602,449, U.S. Pat. No. 7,306,762 and/or U.S. Pat. No. 6,869,563 is shaped in a mold to define at least a portion of a bone structure, and sterilized such that it is configured for implantation as an artificial bone in a human body. For some applications, the ceramic foam material is shaped in a mold to define a block or any other desired shape that is suitable for implantation as a bone prosthesis. Accordingly, the ceramic foam material may be shaped to define any artificial bone configuration required for a subject's specific surgical needs. Additionally or alternatively, the ceramic foam material may be shaped to define a particular three dimensional shape, e.g., as calculated from a subject's CT scan. For some applications, the ceramic foam is machined to define a bone-shaped structure prior to implantation.

For some applications, the ceramic foam material is a generally-rigid solid, having a volume of 0.25-1000 cc, e.g., 0.25-2 cc, 2-100 cc, or 100-1000 cc.

For some applications, the ceramic foam shapes are coated with an external layer, in order to replicate an original bone structure. Additionally or alternatively, the ceramic foam shapes are coated with a coating such as a ceramic coating and/or a chemical coating, and/or a polymeric coating and/or a composite coating. For some applications, the coating facilitates obtaining desired surface proprieties such as hardness and/or selective sealing and/or adhesion enhancement and/or surface roughness. Additionally or alternatively, the coating may affect hydrophobic or hydrophilic proprieties of the bone product. Further additionally or alternatively, the coating may comprise a tribological coating and/or an anti-friction coating to facilitate movement of the artificial bone relative to the native bone, in a joint.

For some applications, the artificial bone is shaped to define channels to facilitate revascularization within the artificial bone. Typically, the channels within the artificial bone are of suitable diameter to allow blood vessels of various diameters (e.g., small-diameter or large-diameter blood vessels) to grow within the channels. For some applications, the channels have a diameter of 5-100 microns and/or 100-500 microns and/or 0.5-10 mm.

For some applications, a bioactive material is added to the ceramic foam material. For applications in which ceramic foam pellets are used, the bioactive material is added to the pellets. For applications in which sintered ceramic foam shapes are used, the active material is added to the foam by impregnation with a liquid solution.

Typically, a growth factor which induces bone and cartilage formation, such as bone morphogenetic proteins, is added to the ceramic foam. Additionally or alternatively, an additional bioactive substance is added to the ceramic foam, e.g., an antibiotic and/or another antibacterial substance, and/or an antiviral agent and/or a hormone. For some applications, adhesion promoters and/or surface modifiers are added to the ceramic foam material. Typically, adhesion promoters facilitate bonding of the artificial bone to native bone and/or tissue. Surface modifiers typically improve the compatibility of the artificial bone within the human body.

For some applications, fillers may be added to the ceramic foam material. For example, the filler may comprise ceramic fibers, polymeric fibers and/or particulate fillers.

Additionally or alternatively, ceramic foam shapes and/or pellets as described herein and in U.S. Pat. Nos. 6,602,449, 7,306,762 and 6,869,563, which are incorporated herein by reference, may be used as bone grafting material. Typically, during and/or after manufacturing by techniques described in U.S. Pat. Nos. 6,602,449, 7,306,762 and 6,869,563, the ceramic foam material, e.g., ceramic foam pellets, are made into bone grafting material suitable for replacement of bone within a human body.

Typically, ceramic foam pellets are used in the manufacturing process of the ceramic foam, as described in U.S. Pat. Nos. 6,602,449, 7,306,762 and 6,869,563, which are incorporated herein by reference. For some applications, in which the ceramic foam is manufactured for use as bone grafting material, a resorbable matrix is added to the ceramic pellets. Typically, the resorbable matrix converts the ceramic pellets into a paste for use as bone grafting material. Additionally or alternatively, additives such as glycerin, hyaluronic acid, and/or polyethylene glycol are added for converting the ceramic pellets into paste.

For some applications, bone graft materials such as beta tricalcium phosphate (beta-TCP) and/or hydroxyapatite are added to the ceramic foam material for use as bone grafting material. Typically, new bone may develop on the bone grafting material; the grafts then slowly dissolve, leaving only the new bone behind. Additionally or alternatively, ceramic pellets of varying sizes (ranging from nanometers to millimeters) may be used in the bone grafting material.

For some applications, a bioactive material such as a growth factor is added to the ceramic foam for use as a bone grafting material. Typically, a growth factor which induces bone and cartilage formation, such as bone morphogenetic proteins, is added to the ceramic foam. Additionally or alternatively, an antibiotic and/or another antibacterial substance is added to the ceramic foam.

For some applications, fillers may be added to the ceramic foam material. For example, fillers may be added during a sintering process in which ceramic pellets are converted into a sintered shape. The filler may comprise ceramic fibers, polymeric fibers, and/or particulate fillers.

For some applications, fillers are added to the ceramic foam material during the manufacturing process as described in U.S. Pat. Nos. 6,602,449, 7,306,762 and/or 6,869,563. As provided by some applications of the present invention, these ceramic foams are heated during sintering of the ceramic foam shapes, in order to destroy the filler fibers, such that channels are formed in their place that allow blood vessels to grow within the channels when used as a bone graft.

The bone grafting material is typically sterilized and packaged in sterile packaging, such that it is suitable for use within the human body.

For some applications, the bone grafting material is injected into the body during a bone grafting procedure.

Typically, the bone grafting material as described herein is generally not resorbable in the body.

Reference is made to the artificial bone and bone grafting ceramic foam materials described herein. Generally, these artificial bone and bone grafting materials are particularly suitable for use in the human body. The ceramic foam materials typically comprise a chemically pure composition substantially devoid of organic content and mineral impurities. Additionally, these ceramic foam materials are generally stable and withstand changing chemical environments (e.g., acidic or basic environments) and are configured for chronic implantation in the body.

Typically, the artificial bone and bone grafting ceramic foam materials described herein have an open-pore microstructure. In some applications, the open-pore microstructure allows penetration of new bone tissue and new blood vessels into the artificial bone and bone grafts. Additionally or alternatively, the open-pore microstructure allows for impregnation of the ceramic foams with functional elements such as the bioactive substances described herein. Additionally, the open-pore microstructure generally allows for enhanced mechanical properties (when compared to other foams of similar density), as well as high-strength adhesion of new bone to the ceramic foam “scaffold,” due to mechanical interlocking.

Additionally or alternatively, the artificial bone and bone grafting ceramic foam materials described herein comprise generally low density ceramic foam, such as 0.3-1.5 g/cc. This typically allows for new bone to grow within the artificial bone and bone grafting structures.

Further additionally, the artificial bone and bone grafting ceramic foam materials described herein generally have a large surface area. The large surface area typically allows for enhanced adhesion of new bone to the ceramic foam implant, facilitating osseointegration. For some applications, the large surface area of the foam materials comprises functional impregnated elements (e.g., bone growth stimulating materials, antibiotics, and/or other drugs) to be impregnated into the ceramic foam.

Typically, the artificial bone and bone grafting ceramic foam materials have generally thin inner walls and voids. Additionally, it is to be noted that the artificial bone and bone grafting ceramic foam materials described herein generally do not trigger an undesired immune response when implanted in the body.

For some applications, the ceramic foam materials described herein are used as a scaffold to grow new tissue in vitro, by shaping the ceramic material into a tissue shape.

For some applications, the ceramic foam materials described herein may be added to a variety of solutions, such as saline hyaluronic acid glycerin and/or other bone graft material, and subsequently injected into the body of the subject.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A sterile artificial bone product, comprising:
a sintered porous ceramic foam material having a density of 0.3 to 1.5 g/cc, and a porosity of 40 to 95%, pores of the material having a pore diameter distribution of 10-500 microns; and
a bioactive compound coupled to the material.

2. The sterile artificial bone product according to claim 1, wherein the ceramic foam material is bone shaped.

3. The sterile artificial bone product according to claim 1, wherein the ceramic foam material is a generally-rigid solid, having a volume of 0.25-1000 cc.

4. The sterile artificial bone product according to claim 1, wherein the sintered porous ceramic foam material comprises alumina.

5. The sterile artificial bone product according to claim 1, wherein the sintered porous ceramic foam material comprises alumina silicate.

6. The sterile artificial bone product according to claim 1, wherein the sintered porous ceramic foam material comprises a ceramic selected from the group consisting of zirconia, alumina zirconate, and alumina titanate.

7. The sterile artificial bone product according to claim 1, further comprising a compound selected from the group of fillers consisting of ceramic fibers, polymeric fibers, and particulate fillers.

8. The sterile artificial bone product according to claim 1, wherein the bone product is shaped to provide a plurality of channels, at least some of the channels having a diameter selected from the group consisting of: 5-100 microns, 100-500 microns and 0.5-10 mm.

9. The sterile artificial bone product according to claim 1, wherein the bioactive compound comprises a substance selected from the group consisting of: a growth factor, an antibiotic, an antibacterial molecule, an antiviral agent, and a hormone.

10. The sterile artificial bone product according to claim 1, further comprising an adhesive promoter

11. The sterile artificial bone product according to claim 1, further comprising a surface modifier.

12. A sterile artificial bone product, comprising:
a material comprising sintered porous ceramic foam pellets having a density of 0.05 to 0.5 g/cc, and a porosity of 70 to 98%, pores of the material having a pore diameter distribution of 10-1000 microns; and
a bioactive compound coupled to the material.

13. The sterile artificial bone product according to claim 12, wherein the sintered porous ceramic foam pellet is bone shaped.

14. The sterile artificial bone product according to claim 12, wherein the material is a generally-rigid solid, having a volume of 0.25-1000 cc.

15. The sterile artificial bone product according to claim 12, wherein the sintered porous ceramic foam pellet comprises alumina.

16. The sterile artificial bone product according to claim 12, wherein the sintered porous ceramic foam pellet comprises alumina silicate.

17. The sterile artificial bone product according to claim 12, wherein the sintered porous ceramic foam pellet comprises a ceramic selected from the group consisting of zirconia, alumina zirconate, and alumina titanate.

18. The sterile artificial bone product according to claim 12, further comprising a compound selected from the group of fillers consisting of ceramic fibers, polymeric fibers, and particulate fillers.

19. The sterile artificial bone product according to claim 12, wherein the bone product is shaped to provide a plurality of channels, at least some of the channels having a diameter selected from the group consisting of: 5-100 microns, 100-500 microns and 0.5-10 mm.

20. The sterile artificial bone product according to claim 12, wherein the bioactive compound comprises a substance selected from the group consisting of: a growth factor, an antibiotic, an antibacterial molecule, an antiviral agent, and a hormone.

21. The sterile artificial bone product according to claim 12, further comprising an adhesive promoter.

22. The sterile artificial bone product according to claim 12, further comprising a surface modifier.

23. The sterile artificial bone product according to claim 12, further comprising a bone grafting material selected from the group consisting of: beta tricalcium phosphate and hydroxyapatite.

24. A method for producing an artificial bone product, comprising:
providing a porous ceramic foam material having a density of 0.3 to 1.5 g/cc, and a porosity of 40 to 95%, pores of the material having a pore diameter distribution of 10-500 microns; and
sintering the ceramic foam material into a bone-shaped mold.

25. The method according to claim 24, wherein sintering the ceramic foam material comprises sintering the ceramic foam material into a mold of volume 0.25-1000 cc.

26. The method according to claim 24, wherein providing the ceramic foam material further comprises providing a ceramic foam material comprising a compound selected from the group of fillers consisting of ceramic fibers, polymeric fibers, and particulate fillers.

27. The method according to claim 26, further comprising creating channels in the ceramic foam material by heating the ceramic foam material sufficiently to destroy the fibers in the ceramic foam material.

28. A method for producing an artificial bone product, comprising:
identifying a desired bone shape; and
machining into the desired bone shape a sintered porous ceramic foam material made from ceramic foamed pellets, the foam material having a density of 0.3 to 1.5 g/cc, and a porosity of to 95%, pores of the material having a pore diameter distribution of 10-500 microns.

29. The method according to claim 28, further comprising coating the desired bone shape after the machining.

30. A method for producing an artificial bone product, comprising:
providing porous ceramic foam pellets having a density of 0.05 to 0.5 g/cc, and a porosity of 70 to 98%, pores of the material having a pore diameter distribution of 10-1000 microns;
sintering the ceramic foam pellets into an artificial bone product; and
packaging the artificial bone product in sterile packaging.
